(19) United States
(12) Patent Application Publication
(10) Pub. No.: US 2026/0176581 A1
(43) Pub. Date: Jun. 25, 2026

(54) PARP INHIBITOR-TOLERANT CAR-T CELLS AND CONSTRUCTION METHODS THEREOF

(71) Applicant: TONGJI HOSPITAL, TONGJI MEDICAL COLLEGE OF HUAZHONG UNIVERSITY OF SCIENCE AND TECHNOLOGY, Wuhan (CN)

(72) Inventors: Yong FANG, Wuhan (CN); Qinglei GAO, Wuhan (CN); Ding MA, Wuhan (CN); Guang HU, Wuhan (CN); Xiaoyuan HUANG, Wuhan (CN); Jiahao LIU, Wuhan (CN); Xiaofei JIAO, Wuhan (CN); Wei MU, Wuhan (CN); Li ZHU, Wuhan (CN)

(73) Assignee: TONGJI HOSPITAL, TONGJI MEDICAL COLLEGE OF HUAZHONG UNIVERSITY OF SCIENCE AND TECHNOLOGY, Wuhan (CN)

(21) Appl. No.: 19/332,208

(22) Filed: Sep. 18, 2025

(30) Foreign Application Priority Data
Dec. 24, 2024 (CN) 202411912183.4

(51) Int. Cl. C12N 5/0783; A61K 35/17; A61K 40/11; A61K 40/31; A61K 40/42; A61P 35/00; C12N 5/10; C12N 9/22; C12N 9/78; C12N 15/113; C12N 15/86

(52) U.S. Cl. CPC C12N 5/0636; A61K 35/17; A61K 40/11; A61K 40/31; A61K 40/42; A61P 35/00; C12N 5/10; C12N 9/226; C12N 9/78; C12N 15/113; C12N 15/86; C12Y 305/04001; C12N 2510/00; C12N 2740/15043

(57) ABSTRACT

A PARP inhibitor-tolerant CAR-T cell and its construction method are provided. A sequence of sgRNA for targeted editing PARP1 gene in CAR-T cells includes sg-D45, sg-S588, or sg-G745. The construction method includes using a cytosine base editor to introduce S588F, G745K and/or D45N mutations into the PARP1 proteins in CAR-T cells with the sgRNA, significantly increasing the tolerance of the CAR-T cells to PARP inhibitors and allowing the CAR-T cells to work synergistically with the PARP inhibitors, thereby maximizing the anti-tumor effect.

### CROSS-REFERENCE TO RELATED APPLICATIONS

This application claims priority to the Chinese patent application No. 202411912183.4 filed on Dec. 24, 2024, the entire contents of which are hereby incorporated by reference.

### SEQUENCE LISTING

The instant application contains a Sequence Listing which is submitted electronically in XML format and is hereby incorporated by reference in its entirety. The XML copy, created on Mar. 25, 2025, is named “2025 Mar. 25-Sequence List-67905-H004US00,” and is 6,472 bytes in size.

### TECHNICAL FIELD

The present disclosure relates to the technical field of tumor immunotherapy, and in particular, to PARP inhibitor-tolerant CAR-T cells and construction methods thereof.

### BACKGROUND

Poly ADP ribose polymerase inhibitor (PARP inhibitor, PARPi) is a targeted drug widely used in cancer treatment, and it is clinically applied to some subtypes of breast cancer, ovarian cancer, prostate cancer, and colorectal cancer. PARPi is also a maintenance therapy drug that can effectively prolong overall survival of patients with ovarian cancer. Clinical studies have demonstrated that PARPi maintenance therapy can significantly delay the recurrence of ovarian cancer and reduce mortality rate. For example, a first-line maintenance therapy of olaparib, a PARPi, can nearly quadruple the median progression-free survival of patients. Consequently, PARPi has quickly become a first-line recommended drug for ovarian cancer in domestic and international guidelines. PARPi drugs such as olaparib, niraparib, rucaparib, and pamiparib have also been approved by the China National Medical Products Administration and U.S. Food and Drug Administration for the treatment of ovarian cancer. However, single-agent PARPi maintenance therapy can only delay disease progression but cannot prevent tumor recurrence. More than 40% of patients will experience recurrence of ovarian cancer within 5 years of PARPi maintenance therapy.

Chimeric antigen receptor T cell (CAR-T) therapy is one of the most advanced and promising immunotherapies. It primarily involves the tumor-specific and targeted modification of the patients' T cells to enable the modified T cells to specifically recognize and kill tumor cells. Due to inherent ability of the CAR-T cells to survive long-term and autonomously expand in vivo, the CAR-T therapy exhibits a distinct advantage in the long-term tumor surveillance and prolonging survival of patients. The combination of PARPi and CAR-T may provide a new approach to achieving these objectives. However, PARPi exerts a strong toxicity to CAR-T cells, for example, treatment with olaparib at the maximum plasma concentration (Cmax) for 72 hours can inhibit CAR-T cells by more than 80%. This toxicity poses a challenge to fully realize the potential of the combined CAR-T and PARP therapy.

Therefore, it is desired to provide a PARP inhibitor-tolerant CAR-T cell.

### SUMMARY

One or more embodiments of the present disclosure provide an sgRNA for targeted editing of a PARP1 gene in CAR-T cells, comprising a sequence selected from a group consisting of sg-D45, sg-S588, and sg-G745; wherein a nucleotide sequence of the sg-D45 is shown in SEQ ID NO.1, a nucleotide sequence of the sg-S588 is shown in SEQ ID NO.2, and a nucleotide sequence of the sg-G745 is shown in SEQ ID NO.3.

One or more embodiments of the present disclosure provide a PARP inhibitor-tolerant CAR-T cell, wherein the PARP inhibitor-tolerant CAR-T cell is constructed using the sg-D45, the sg-S588, or the sg-G745.

In some embodiments, an amino acid at position 588 of a PARP1 protein in the PARP inhibitor-tolerant CAR-T cell is mutated from S to F, or an amino acid at position 745 of the PARP1 protein in the PARP inhibitor-tolerant CAR-T cell is mutated from G to K, or an amino acid at position 45 of the PARP1 protein in the PARP inhibitor-tolerant CAR-T cell is mutated from D to N.

One or more embodiments of the present disclosure provide a method for constructing a PARP inhibitor-tolerant CAR-T cell, comprising: using a cytosine base editor to edit genes encoding PARP1 proteins in T cells with the sgRNA, and obtaining the PARP inhibitor-resistant CAR-T cell.

In some embodiments, the method further comprises: activating the T cells; using an electroporation reagent to transfer the sgRNA and CBE3 protein into the activated T cells via electroporation; and adding CAR lentivirus to a culture system of the T cells for transfection and culture to obtain the PARP inhibitor-tolerant CAR-T cell.

In some embodiments, the CBE3 protein is a fusion protein of nCas9 protein and cytosine deaminase.

In some embodiments, the electroporation reagent is a Lonza P3 Primary Cell 4D-Nucleofector™ X Kit.

In some embodiments, the electroporation is performed on the T cells 24 hours after activation, and the transfection is performed 6 hours after the electroporation.

One or more embodiments of the present disclosure provide a synergistic anti-tumor agent, wherein the synergistic anti-tumor agent comprises the above-mentioned PARP inhibitor-tolerant CAR-T cell.

### BRIEF DESCRIPTION OF THE DRAWINGS

The present disclosure will be further illustrated by way of exemplary embodiments, which will be described in detail through the accompanying drawings, wherein:

FIG. 1 is a schematic diagram illustrating a construction process of PARP inhibitor-tolerant CAR-T cells according to some embodiments of the present disclosure;

FIG. 2 is a schematic diagram illustrating expression levels of CAR molecules of PARP inhibitor-tolerant CAR-T cells according to some embodiments of the present disclosure;

FIG. 3 is a schematic diagram illustrating gene editing characteristics of genes encoding PARP1 proteins in PARP inhibitor-tolerant CAR-T cells according to some embodiments of the present disclosure;

FIG. 4 is a diagram illustrating cell activity of PARP inhibitor-tolerant CAR-T cells at different concentrations of olaparib according to some embodiments of the present disclosure;

FIG. 5 is a diagram illustrating cell activity of PARP inhibitor-tolerant CAR-T cells at a concentration of 25 UM of olaparib according to some embodiments of the present disclosure;

FIG. 6 is a diagram illustrating a change in fluorescence intensity of tumor cells treated with PARP inhibitor-tolerant CAR-T cells at different concentrations of olaparib according to some embodiments of the present disclosure;

FIG. 7 is a diagram illustrating specific lysis of tumor cells by PARP inhibitor-tolerant CAR-T cells at different concentrations of olaparib according to some embodiments of the present disclosure;

FIG. 8 is a diagram illustrating an effect of PARP inhibitor-tolerant CAR-T cells on tumor volume in mice bearing a SKOV3 model according to some embodiments of the present disclosure;

FIG. 9 is a diagram illustrating an effect of PARP inhibitor-tolerant CAR-T cells on tumor volume in mice bearing a patient-derived xenograft model of ovarian cancer according to some embodiments of the present disclosure; and

FIGS. 10A-10G are diagrams illustrating an effect of PARP inhibitor-tolerant CAR-T cells on tumor volume in mice bearing a patient-derived xenograft model of ovarian cancer and tumor infiltration of CAR-T cells according to some embodiments of the present disclosure.

### DETAILED DESCRIPTION

In order to provide a clearer understanding of the technical solutions of the embodiments described in the present disclosure, a brief introduction to the drawings required in the description of the embodiments is given below. It is evident that the drawings described below are merely some examples or embodiments of the present disclosure, and for those skilled in the art, the present disclosure may be applied to other similar situations without exercising creative labor, unless otherwise indicated or stated in the context, the same reference numerals in the drawings represent the same structures or operations.

As set forth in the present disclosure and the claims, unless explicitly indicated otherwise in the context, words such as “one”, “a”, “an”, and/or “the” do not specifically denote the singular form and may also include the plural form. In general, the terms “comprising” and “including” only suggest the inclusion of steps and elements that have been explicitly identified, and these steps and elements do not constitute an exclusive listing; methods may also include other steps or elements.

Unless otherwise defined, all technical and scientific terms used in the present disclosure have the same meaning as typically understood by those of ordinary skill in the art to which the present disclosure pertains.

One or more embodiments of the present disclosure provide an sgRNA for targeted editing of a PARP1 gene in CAR-T cells, comprising a sequence selected from a group consisting of sg-D45, sg-S588, and sg-G745; a nucleotide sequence of the sg-D45 is shown in SEQ ID NO. 1, a nucleotide sequence of the sg-S588 is shown in SEQ ID NO. 2, and a nucleotide sequence of the sg-G745 is shown in SEQ ID NO. 3.

One or more embodiments of the present disclosure provide a PARP inhibitor-tolerant CAR-T cell, and the PARP inhibitor-tolerant CAR-T cell is constructed using the sg-D45, the sg-S588, or the sg-G745.

In some embodiments, an amino acid at position 588 of a PARP1 protein in the PARP inhibitor-tolerant CAR-T cell is mutated from S to F, or an amino acid at position 745 of the PARP1 protein in the PARP inhibitor-tolerant CAR-T cell is mutated from G to K, or an amino acid at position 45 of the PARP1 protein in the PARP inhibitor-tolerant CAR-T cell is mutated from D to N.

In some embodiments, in the PARP inhibitor-tolerant CAR-T cell, the nucleotide sequence encoding the PARP1 protein includes a nucleotide sequence encoding CART-T_{D45}, a nucleotide sequence encoding CART-T_{S588}, or a nucleotide sequence encoding CART-T_{G745}. The nucleotide sequence encoding CART-T_{D45} is shown as SEQ ID NO. 4 (ATCAAACATGGGCGACTAGAAGG), the nucleotide sequence encoding CART-T_{S588} is shown as SEQ ID NO. 5 (GTCCTGGGGCCGTGTGGGTACGG), and the nucleotide sequence encoding CART-T_{G745} is shown as SEQ ID NO. 6 (GGGACTAGGGGGTGCTGAAACCC).

One or more embodiments of present disclosure provide a method for constructing the above-mentioned PARP inhibitor-resistant CAR-T cells, including using a cytosine base editor (CBE) to edit the genes encoding the PARP1 proteins in T cells with the above-mentioned sgRNA, and obtaining the PARP inhibitor-resistant CAR-T cell.

The principle of the cytosine base editor involves fusing the nCas9 protein, which can only cleave single-stranded DNA to produce nicks, with a cytosine deaminase to generate a CBE3 protein. With the guidance of the sgRNA, the base-editing enzyme (CBE3 protein) is precisely anchored to the DNA target site. After CBE3 binds to the DNA target site, the sgRNA and the target-site DNA pair form an “R-loop” through complementary base-pairing, so as to generate local single-stranded DNA (ssDNA). The cytosine deaminase deaminates cytosine (C) within a specific range of the ssDNA in the “R-loop”, thereby converting C into uracil (U). Through the DNA repair or replication process, U is further converted into thymine (T), ultimately achieving the direct substitution of C to T or G to A.

In some embodiments, the method further comprises: activating the T cells; using an electroporation reagent to transfer the sgRNA and CBE3 protein into the activated T cells via electroporation; and adding CAR lentivirus to a culture system of the T cells for transfection and culture to obtain the PARP inhibitor-tolerant CAR-T cell.

In some embodiments, the CBE3 protein is a fusion protein of nCas9 protein and cytosine deaminase.

In some embodiments, the electroporation reagent is a Lonza P3 Primary Cell 4D-Nucleofector™ X Kit.

In some embodiments, the electroporation is performed on the T cells 24 hours after activation, and the transfection is performed 6 hours after the electroporation.

One or more embodiments of the present disclosure provide use of the PARP inhibitor-tolerant CAR-T cell, and the PARP inhibitor-tolerant CAR-T cell is used for preparation of synergistic anti-tumor agent.

One or more embodiments of the present disclosure provide a synergistic anti-tumor agent, and the synergistic anti-tumor agent comprises the above-mentioned PARP inhibitor-tolerant CAR-T cell.

The embodiments of the present disclosure have at least the following beneficial effects: by introducing S588F, G745K and D45N mutations in the PARP1 proteins in CAR-T cells via CBE, the tolerance of CAR-T cells to PARP inhibitors can be significantly increased. When PARP inhibitor-tolerant CAR-T cells are combined with PARP inhibitor for therapy, they exhibit excellent anti-tumor efficacy. The PARP inhibitor-tolerant CAR-T cells obtained through the construction method of the embodiments of the present disclosure have high gene editing efficiency and strong infectivity. After treatment with PARP inhibitors, DNA damage is significantly reduced, and proliferation inhibition is also greatly decreased. At the same time, these PARP inhibitor-tolerant CAR-T cells have significant PARP inhibitor tolerance effects both in vitro and in vivo. The PARP inhibitor-tolerant CAR-T cells provided by the embodiments of the present disclosure are obtained by performing targeted modification on existing CAR-T cells, enabling the modified CAR-T cells to tolerate the toxicity of PARP inhibitors. As a result, the modified CAR-T cells can be used in combination with PARP inhibitors, thereby maximizing the anti-tumor effect.

The experimental techniques in the following examples, unless otherwise specified, are conventional techniques. The experimental materials used in the following examples, unless otherwise specified, are obtained from standard biochemical reagent companies. Quantitative assays in the following examples are performed with three replicate experiments, and the results are averaged. It should be understood that the following examples are for better explaining the present disclosure and are not intended to limit the scope of the present disclosure.

### EXAMPLES

### Example 1: Construction of PARP Inhibitor-Tolerant CAR-T Cells

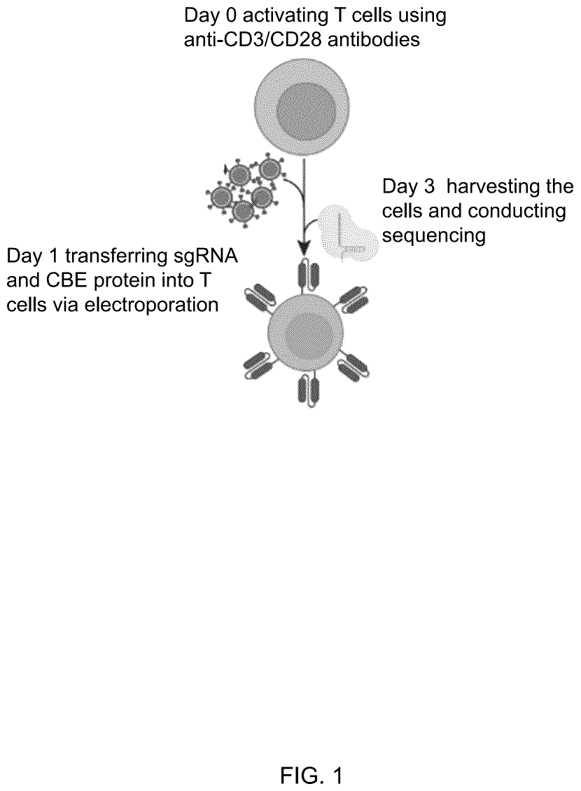

The construction process of the PARP inhibitor-tolerant CAR-T cells in the example is shown in FIG. 1, including following operations.

The required amount of T cells was taken and activated with anti-CD3/CD28 antibodies. Electroporation was performed 24 hours after the activation to transfer the GenScript EasyEdit sgRNA and CBE3 protein into the activated T cells via the EH115 program of the Lonza 4D-Nucleofector electroporator. The electroporation reagent was Lonza P3 Primary Cell 4D-Nucleofector™ X Kit, and the CBE3 protein was commercially available.

The Sequence of sgRNA Includes:

SEQ ID NO. 1 (sg-D45): CAAACATGGGCGACTAGAAGG;
SEQ ID NO. 2 (sg-S588): CCTGGGGCCGTGTGGGTACGG;
SEQ ID NO. 3 (sg-G745): GGGACTAGGGGGTGCTGAAACCC.

CAR lentivirus was added to the culture system 6 hours after electroporation for transfection. The cells were harvested 3 days after electroporation, and DNA was extracted for site-specific amplification, followed Sanger sequencing.

EditR was used to analyze the Sanger sequencing results, and the program could estimate the editing efficiency based on the Sanger sequencing chromatogram while assessing the expression of CAR molecule.

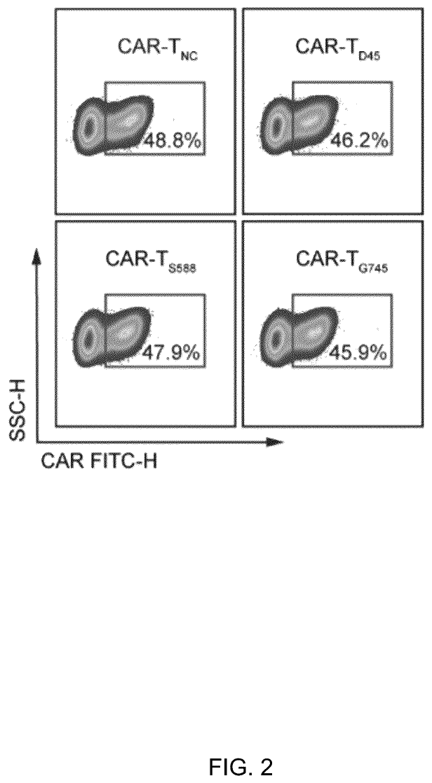

The expression level of the CAR molecule is as shown in FIG. 2. In FIG. 2, SSC-H represents the intensity of the side-scatter light signal; CAR FITC-H represents the intensity of the fluorescence signal of the CAR molecule labeled with FITC. As can be seen from FIG. 2, the S588F (CAR-T_{S588}), G745K (CAR-T_{G745}), and D45N (CAR-T_{D45}) mutations introduced into the PARP1 proteins in CAR-T cells all have very high expression levels, indicating that the PARP1 gene editing does not affect the CAR expression level.

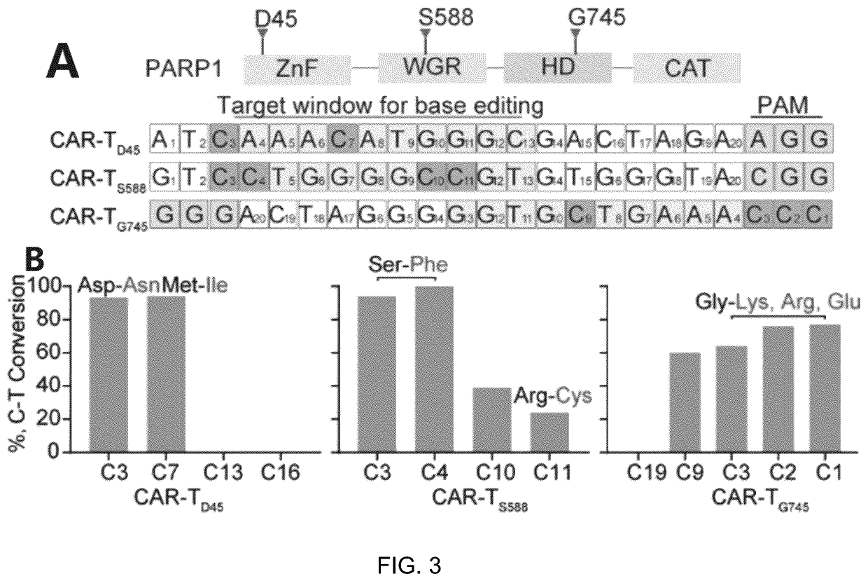

The gene editing characteristics of the gene encoding the PARP1 protein in the PARP inhibitor-resistant CAR-T cells are as shown in FIG. 3. In FIG. 3, A is a schematic diagram of the mutation sites and base structure of the gene encoding the PARP1 protein, and B is the base conversion rate of each mutation of the gene encoding the PARP1 protein. According to FIG. 3, it can be seen that the constructed CAR-T cells have an excellent gene-editing efficiency.

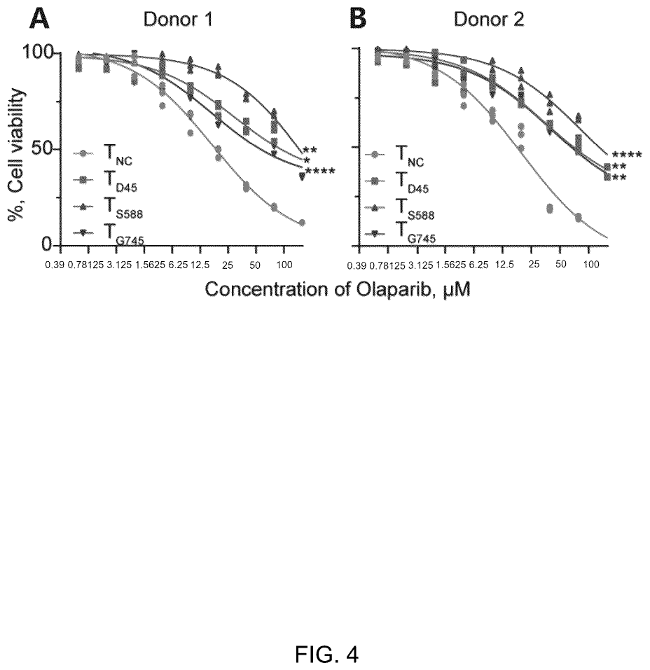

The AOPI dye was used to estimate the number of surviving CAR-T cells after treatment with different concentrations of PARPi (0.39, 0.78125, 1.5625, 3.125, 6.25, 12.5, 25, 50, and 100 μM) according to the proportion of live cells and the total number of cells. A proliferation curve was drawn, and the inhibition rate of PARPi was calculated by comparison with the control group (CAR-T_{NC}). The IC50 was estimated using Prism9 software. Two test groups were set up in the example. The experimental results of the first test group (Donor 1) are as shown in A of FIG. 4, and the experimental results of the second test group (Donor 2) are as shown in B of FIG. 4.

### Example 2: Changes in Cell Activity, DNA Damage, and Apoptosis Levels of PARP Inhibitor-Tolerant CAR-T Cells after Treatment with PARP Inhibitors

The required amount of T cells was taken and activated. Electroporation was performed 24 hours after activation to transfer GenScript EasyEdit sgRNA and CBE3 protein (25 μg) into 1×10⁶ T cells via the EH115 program of the Lonza 4D-Nucleofector electroporator. The electroporation reagent was the Lonza P3 Primary Cell 4D-Nucleofector™ X Kit. CAR lentivirus was added to the culture system 6 hours after electroporation for transfection. After transfection, the cells were cultured and expanded using a complete T-cell medium (OpTmizer CTS T Cell Expansion Basal Medium+200 IU/mL human IL-2+50 ng/ml human IL-15+40 ng/ml human IL-7+1% L-Glutamine+10% FBS). The cells were harvested 72 hours after transfection. A portion of the cells was subjected to flow cytometry to detect the proportion of T cells successfully expressing CAR (CAR⁺ T cells), and another portion of the cells was used for Sanger sequencing to assess the gene editing efficiency. The CAR-T cells edited with sg-D45, sg-S588, and sg-G745 were named CAR-T_{D45}, CAR-T_{S588}, and CAR-T_{G745} respectively.

The PARP inhibitor-tolerant CAR-T cells (CAR-T_{D45}, CAR-T_{S588}, and CAR-T_{G745}) were co-cultured with SKOV3 (SKOV3-Luci), OVCAR3 (OVCAR3-Luci), and OVCAR8 (OVCAR8-Luci) tumor cells. An opaque 96-well plate was seeded with 20,000 tumor cells per well. The PARP inhibitor-resistant CAR-T cells were added according to the effector cell-to-target cell ratios (E:T ratios) of 2:1, 1:1, 1:2, 1:4, 1:8, and 1:16. Untreated CAR-T cells (CAR-T_{NC}) were used as the control group. PARPi was added to the culture system at a specified concentration. The concentration (Cmax) of olaparib was 25 μM. After 24 hours of co-culture, the fluorescence intensity of tumor cells was recorded using a multifunctional enzyme marker and compared with the control group in order to assess the amount of remaining tumor cells in the culture system, thereby evaluating the cytotoxicity of PARP inhibitor-tolerant CAR-T cells combined with PARP inhibitors on tumor cells in vitro.

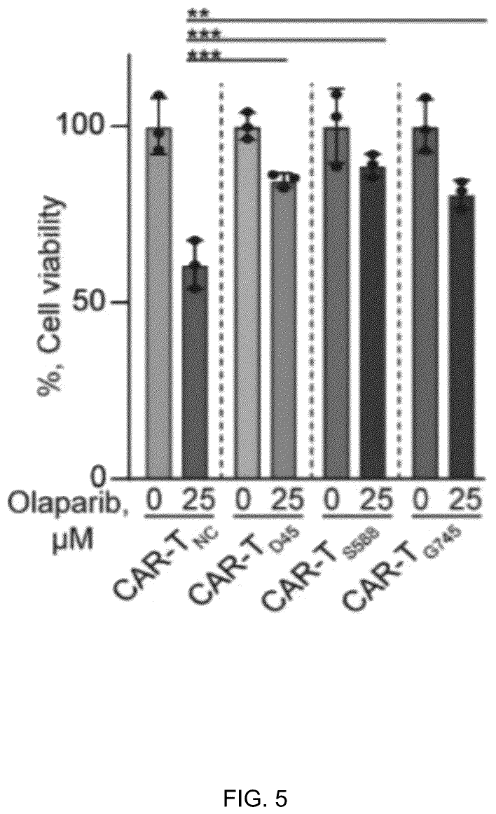

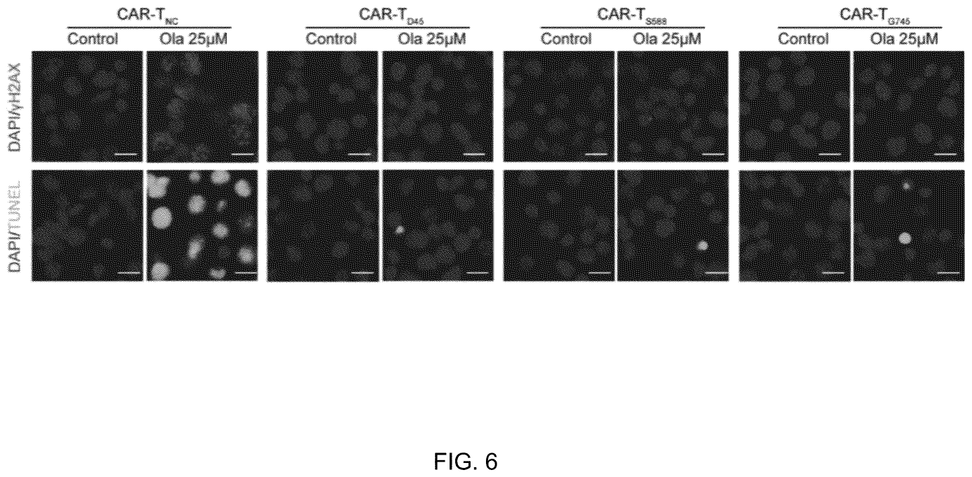

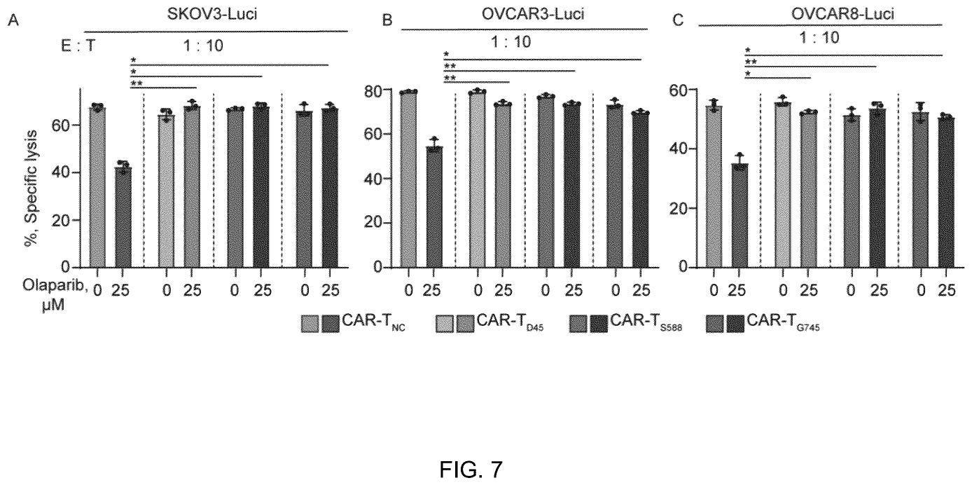

The experimental results are shown in FIGS. 5 to 7. It can be seen from FIGS. 5 to 7 that PARP inhibitor-tolerant CAR-T cells exhibit significant tolerance to the PARP inhibitors.

### Example 3: Anti-Tumor Efficacy of PARP Inhibitor-Tolerant CAR-T Cells Combined with PARP Inhibitors in an In Vitro Model

In this example, NCG mice were used as the mouse model, and two types of xenograft tumor models were constructed: one was a SKOV3-derived xenograft tumor model based on the SKOV3 ovarian cancer cell line, and the other was a patient-derived xenograft tumor model based on the tumor tissue of ovarian cancer patients. The specific operation steps were as follows. For the SKOV3-derived xenograft model, 5×10⁶ SKOV3 tumor cells were injected subcutaneously into the mice; when the tumor size grew to 100-200 mm³, the mice were treated with PARP inhibitor-resistant CAR-T cells combined with PARP inhibitors; mouse blood was collected weekly, the tumor volume and mouse body weight were measured every three days, and the tumor tissues were collected after consecutive administration for 28 days. For the patient-derived xenograft tumor model, tumor tissues of 3 mm×3 mm were transplanted subcutaneously into the lateral wall of the mice; when the tumor size grew to 100-200 mm³, the mice were treated with PARP inhibitor-resistant CAR-T cells combined with PARP inhibitors; mouse blood was collected weekly, the tumor volume and mouse body weight were measured every three days, and the tumor tissues were collected after consecutive administration for 28 days.

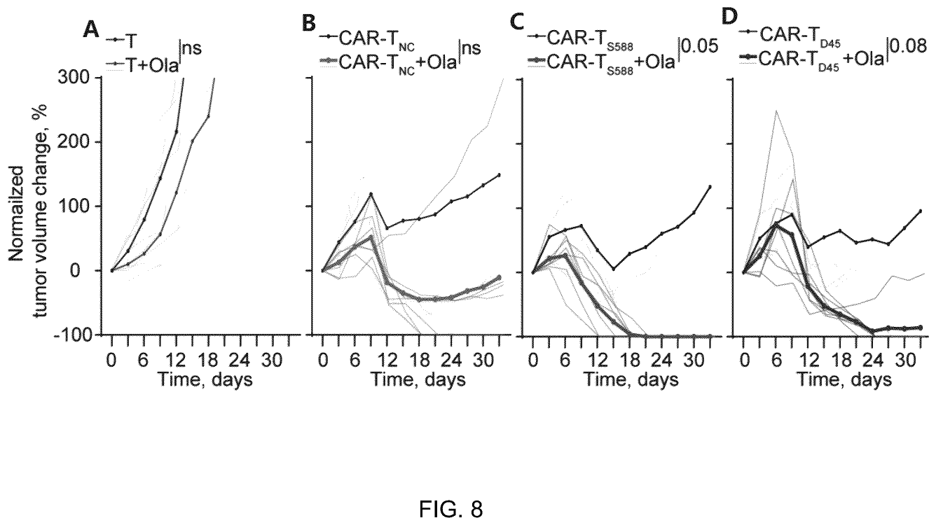

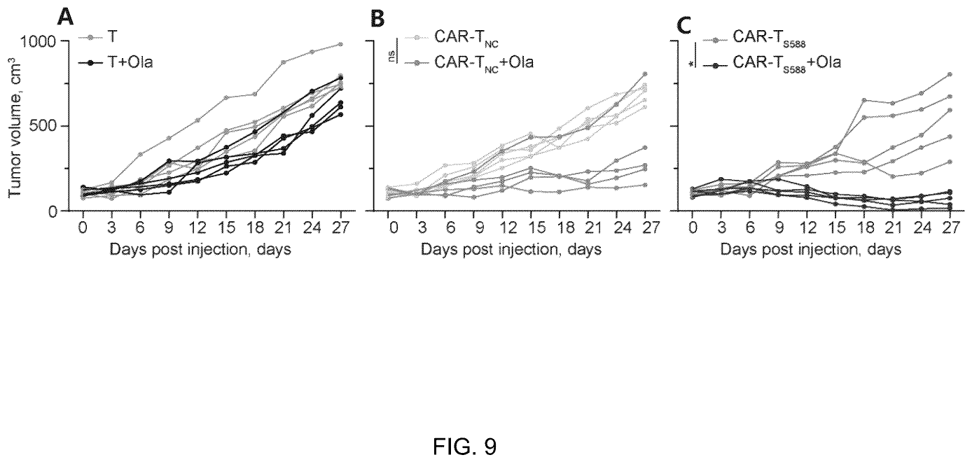

In the SKOV3-derived xenograft tumor model, the tumor volume change in the mice of each experimental group are as shown in FIG. 8. In FIG. 8, A, B, C, and D respectively represent the tumor volume change in the mice after treatment with T cells (T) and T combined with olaparib (T+Ola), unedited CAR-T cells (CAR-T_{NC}) and CAR-T_{NC} combined with olaparib (CAR-T_{NC}+Ola), CAR-T_{S588} and CAR-T_{S588} combined with olaparib (CAR-T_{S588}+Ola), and CAR-T_{D45} and CAR-T_{D45} combined with olaparib (CAR-T_{D45}+Ola). In the patient-derived xenograft tumor model, the tumor volume change in mice of each experimental group is as shown in FIG. 9. In FIG. 9, A, B, and C represent the tumor volume change in mice after treatment with T and T+Ola, CAR-T_{NC} and CAR-T_{NC}+Ola, and CAR-T_{S588} and CAR-T_{S588}+Ola. It could be seen that in the above two models, the combination of PARP inhibitor-resistant CAR-T cells and PARP inhibitors exhibits a significant inhibitory effect on tumors in vivo.

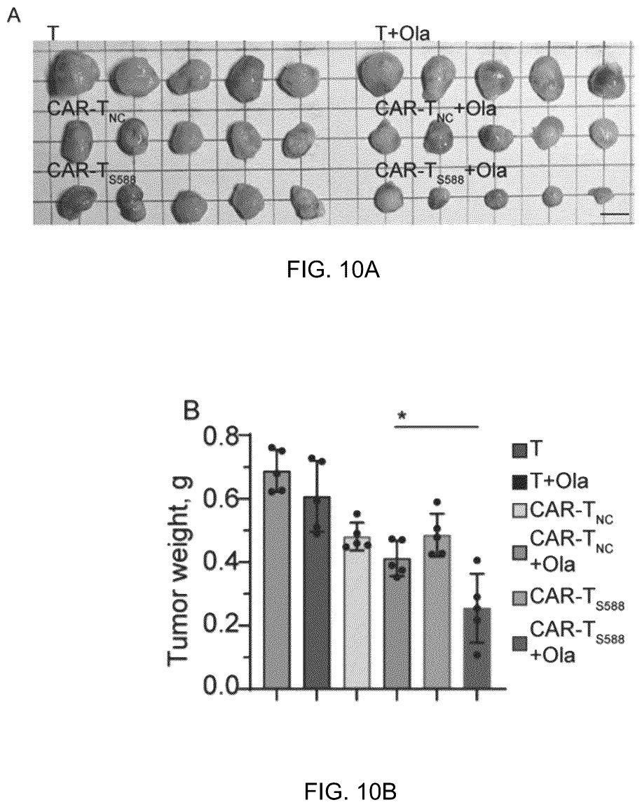

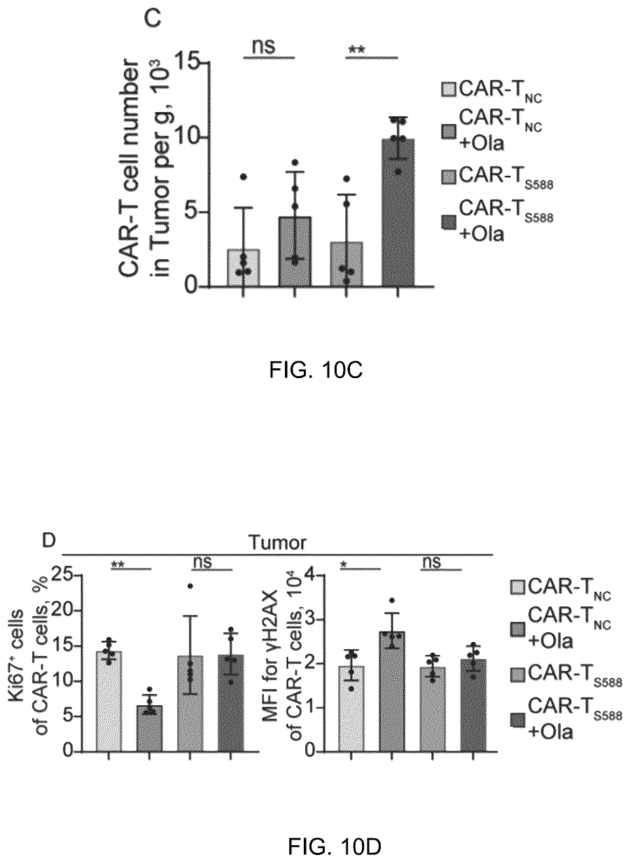

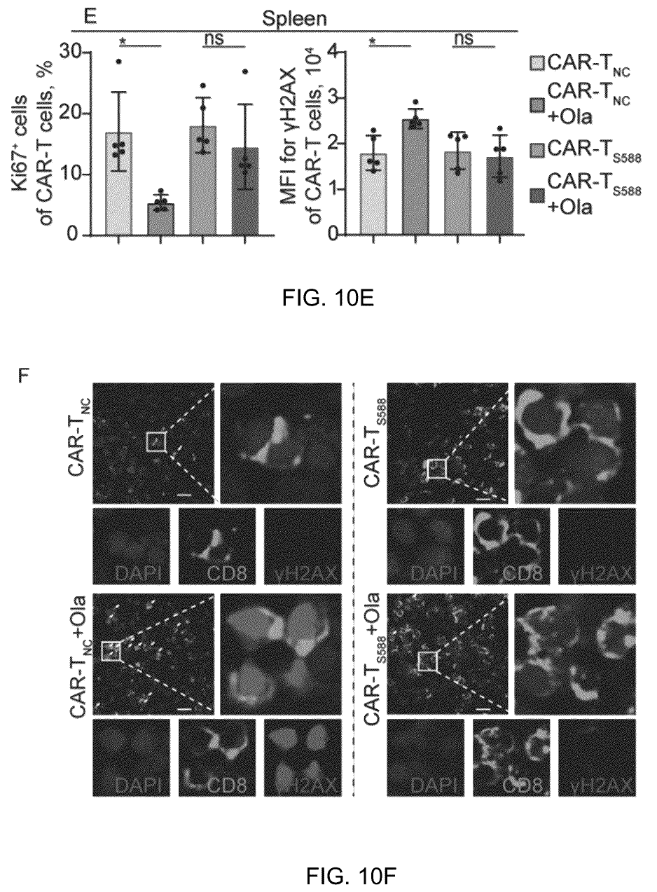

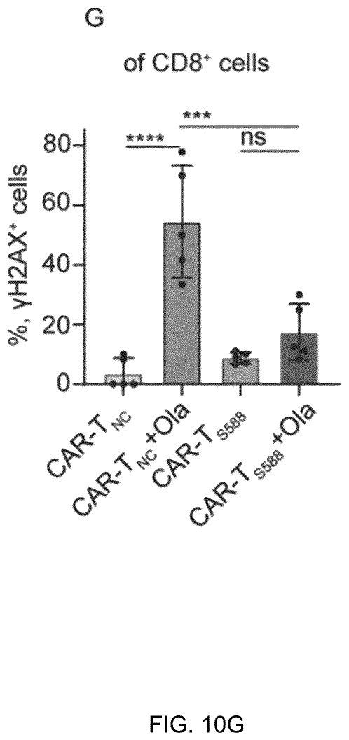

In addition, in order to further evaluate the tumor inhibitory effect of CAR-T_{S588}, the following tests were also carried out in the example.
(1) The anti-tumor activity of PARP inhibitor-tolerant CAR-T cells combined with PARPi was evaluated by comparing the tumor growth curves of different groups and the tumor weight of different groups at the end of the experiment. The experimental results are as shown in FIG. 10A and FIG. 10B. Specifically, FIG. 10A shows the tumor volume change after treatment with T and T+Ola, CAR-T_{NC} and CAR-T_{NC}+Ola, and CAR-T_{S588} and CAR-T_{S588}+Ola, respectively, and FIG. 10B illustrates the comparison of tumor weight of each group.
(2) Multicolor flow cytometry was used to detect the infiltration level of CAR-T cells within tumor cells. The specific procedure was as follows: after collecting the tumors, tumor tissues of the same weight were taken for digestion and antibody labeling, the staining panel included live/dead-aqua, CD45-APCCY7, CD3-PRECP-CY5.5, CD4-BV785, CD8-BV650, and CAR-FITC, and the infiltration level of CAR-T cells in the tumor was reflected by the number of CAR⁺ T cells. The experimental results are shown in FIGS. 10C-10E. Specifically, FIG. 10C illustrates the number of tumor-infiltrating CAR-T and CAR-T_{S588} cells detected by flow cytometry, FIG. 10D illustrates the expression levels of KI67 and γH2AX in tumor-infiltrating CAR-T and CAR-T_{S588} cells, and FIG. 10E illustrates the expression levels of KI67 and γH2AX in CAR-T cells in the mouse spleen.
(3) Multicolor immunohistochemistry was used to detect the infiltration level of CAR-T within tumor cells. The specific procedure was as follows: after collecting the tumor, tissue embedding and multiplex immunohistochemical staining were performed, the staining indicators included CD3, CD8, and γH2AX. The experimental results are shown in FIG. 10F and FIG. 10G. Specifically, FIG. 10F illustrates the results of multicolor immunohistochemical staining of the expression levels of KI67 and γH2AX in tumor-infiltrating CAR-T and CAR-T_{S588} cells, and FIG. 10G is the statistical chart of FIG. 10F.

According to the above experimental results, it can be seen that compared with ordinary CAR-T cells, the PARP inhibitor-tolerant CAR-T cells in the embodiments of the present disclosure exhibits significantly reduced DNA damage and less proliferation inhibition after treatment with PARP inhibitors. The PARP inhibitor-tolerant CAR-T cells demonstrates a good synergistic therapeutic effect when combined with PARP inhibitors for the treatment of ovarian cancer.

The basic concepts have been described above, apparently, in detail, as will be described above, and do not constitute limitations of the disclosure. Although there is no clear explanation here, those skilled in the art may make various modifications, improvements, and corrections of the present disclosure. This type of modifications, improvements, and corrections are recommended in the present disclosure, so the modifications, improvements, and the corrections remain in the spirit and scope of the exemplary embodiment of the present disclosure.

At the same time, the present disclosure uses specific words to describe the embodiments of the present disclosure. As “one embodiment”, “an embodiment”, and/or “some embodiments” means a certain feature, structure, or characteristic of at least one embodiment of the present disclosure. Therefore, it is emphasized and should be appreciated that two or more references to “an embodiment” or “one embodiment” or “an alternative embodiment” in various parts of the present disclosure are not necessarily all referring to the same embodiment. Further, certain features, structures, or features of one or more embodiments of the present disclosure may be combined.

In addition, unless clearly stated in the claims, the order of processing elements and sequences, the use of numbers and letters, or the use of other names in the present disclosure are not used to limit the order of the procedures and methods of the present disclosure. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the subject matter of the present disclosure object requires more features than the features mentioned in the claims. Rather, the claimed subject matter may lie in less than all features of a single foregoing disclosed embodiment.

In some embodiments, the numbers expressing quantities of ingredients, properties, and so forth, used to describe and claim certain embodiments of the application are to be understood as being modified in some instances by the terms “about”, “approximate”, or “substantially”. Unless otherwise stated, “about”, “approximate”, or “substantially” may indicate ±20% variation of the value it describes. Accordingly, in some embodiments, the numerical parameters used in the disclosure and claims are approximate values, and the approximation may change according to the characteristics required by the individual embodiments. In some embodiments, the numerical parameter should consider the prescribed effective digits and adopt a general digit retention method. Although in some embodiments, the numerical fields and parameters used to confirm the breadth of its range are approximate values, in specific embodiments, such numerical values are set as accurately as possible within the feasible range.

With respect to each patent, patent application, patent application disclosure, and other material cited in the present disclosure, such as articles, books, manuals, publications, documents, etc., the entire contents thereof are hereby incorporated by reference into the present disclosure. Application history documents that are inconsistent with the contents of the present disclosure or that create conflicts are excluded, as are documents (currently or hereafter appended to the present disclosure) that limit the broadest scope of the claims of the present disclosure. It should be noted that in the event of any inconsistency or conflict between the descriptions, definitions, and/or use of terms in the materials appended to the present disclosure and those described in the present disclosure, the descriptions, definitions, and/or use of terms in the present disclosure shall prevail.

At last, it should be understood that the embodiments described in the present disclosure are merely illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. An sgRNA for targeted editing of a PARP1 gene in CAR-T cells, comprising a sequence selected from a group consisting of sg-D45, sg-S588, and sg-G745; wherein
a nucleotide sequence of the sg-D45 is shown in SEQ ID NO.1, a nucleotide sequence of the sg-S588 is shown in SEQ ID NO.2, and a nucleotide sequence of the sg-G745 is shown in SEQ ID NO.3.

2. A PARP inhibitor-tolerant CAR-T cell, wherein the PARP inhibitor-tolerant CAR-T cell is constructed using the sg-D45, the sg-S588, or the sg-G745 of claim 1.

3. The cell of claim 2, wherein an amino acid at position 588 of a PARP1 protein in the cell is mutated from S to F, or an amino acid at position 745 of the PARP1 protein in the cell is mutated from G to K, or an amino acid at position 45 of the PARP1 protein in the cell is mutated from D to N.

4. A method for constructing a PARP inhibitor-tolerant CAR-T cell, comprising:
using a cytosine base editor to edit genes encoding a PARP1 protein in T cells with the sgRNA of claim 1, and
obtaining the PARP inhibitor-resistant CAR-T cell.

5. The method of claim 4, comprising:
activating the T cells;
using an electroporation reagent to transfer the sgRNA and CBE3 protein into the activated T cells via electroporation; and
adding CAR lentivirus to a culture system of the T cells for transfection and culture to obtain the PARP inhibitor-tolerant CAR-T cell.

6. The method of claim 5, wherein the CBE3 protein is a fusion protein of nCas9 protein and cytosine deaminase.

7. The method of claim 5, wherein the electroporation reagent is a Lonza P3 Primary Cell 4D-Nucleofector™ X Kit.

8. The method of claim 5, wherein the electroporation is performed on the T cells 24 hours after activation, and the transfection is performed 6 hours after the electroporation.

9. A synergistic anti-tumor agent, wherein the synergistic anti-tumor agent comprises the PARP inhibitor-tolerant CAR-T cell of claim 2.


SEQUENCE LISTING (6 sequences)

SEQ ID NO: 1 - DNA, 21 bp, synthetic construct
  source 1..21: mol_type = other DNA
caaacatgggcgactagaagg

SEQ ID NO: 2 - DNA, 21 bp, synthetic construct
  source 1..21: mol_type = other DNA
cctggggccgtgtgggtacgg

SEQ ID NO: 3 - DNA, 23 bp, synthetic construct
  source 1..23: mol_type = other DNA
gggactagggggtgctgaaaccc

SEQ ID NO: 4 - DNA, 23 bp, synthetic construct
  source 1..23: mol_type = other DNA
atcaaacatgggcgactagaagg

SEQ ID NO: 5 - DNA, 23 bp, synthetic construct
  source 1..23: mol_type = other DNA
gtcctggggccgtgtgggtacgg

SEQ ID NO: 6 - DNA, 23 bp, synthetic construct
  source 1..23: mol_type = other DNA
gggactagggggtgctgaaaccc